(19) United States
(12) Patent Application Publication
(10) Pub. No.: US 2023/0310327 A1
(43) Pub. Date: Oct. 5, 2023

(54) PHARMACEUTICAL COMPOSITIONS COMPRISING RIBOCICLIB

(71) Applicant: NATCO PHARMA LIMITED, Hyderabad (IN)

(72) Inventors: Krishna Murthy BHAVANASI, Hyderabad (IN); Rama Swamy Chowdary Tripuraneni, Hyderabad (IN); Chaitanya Kumar SAGI, Hyderabad (IN); Pavan BHAT, Hyderabad (IN); Venkaiah Chowdary NANNAPANENI, Hyderabad (IN)

(21) Appl. No.: 18/019,209

(22) PCT Filed: Jul. 31, 2021
(86) PCT No.: PCT/IN2021/050740

§ 371 Date: Feb. 1, 2023

(30) Foreign Application Priority Data
Aug. 3, 2020 (IN) 202041033199

(51) Int. Cl. A61K 9/28; A61K 31/519

(52) U.S. Cl. CPC A61K 9/2866; A61K 31/519; A61K 9/2893

(57) ABSTRACT

The present invention relates to pharmaceutical compositions comprising a kinase inhibitor. More particularly, the present invention relates to immediate release pharmaceutical compositions comprising Ribociclib or a pharmaceutically acceptable salt thereof and one or more pharmaceutically acceptable excipients and process for preparing such compositions.

### FIELD OF THE INVENTION

The present invention relates to pharmaceutical compositions comprising a kinase inhibitor. More particularly, the present invention relates to immediate release pharmaceutical compositions comprising Ribociclib or a pharmaceutically acceptable salt thereof and one or more pharmaceutically acceptable excipients and process for preparing such compositions.

### BACKGROUND OF THE INVENTION

Ribociclib is a kinase inhibitor and is approved in the form of succinate salt. Ribociclib succinate is chemically known as butanedioic acid-7-cyclopentyl-N,N-dimethyl-2-{[5-(piperazin-1-yl)pyridin-2-yl]amino}-7H-pyrrolo[2,3-d]-pyrimidine-6-carboxamide (1/1).

Ribociclib in the form of succinate salt is approved in the form of 200 mg tablets and marketed by Novartis under the brand name KISQALI®.

Ribociclib is indicated in combination with:
an aromatase inhibitor for the treatment of pre/perimenopausal or postmenopausal women, with hormone receptor (HR)-positive, human epidermal growth factor receptor 2 (HER2)-negative advanced or metastatic breast cancer, as initial endocrine-based therapy; or
Fulvestrant for the treatment of postmenopausal women with HR-positive, HER2-negative advanced or metastatic breast cancer, as initial endocrine based therapy or following disease progression on endocrine therapy.

WO 2007/140222 A2 and WO 2010/020675 A1 discloses Ribociclib or a pharmaceutically acceptable salt thereof and further discloses a method for the treatment of cancer by inhibiting of a cyclin-dependent kinase (CDK) comprising administration of an effective amount of ribociclib or a pharmaceutically acceptable salt thereof to a subject in need of treatment thereof.

WO 2012/064805 A1 discloses the succinate salt of Ribociclib in crystalline form.

WO 2016/166703 A1 discloses a coated pharmaceutical oral tablet comprising Ribociclib or its pharmaceutically acceptable salt wherein the coating comprises polyvinyl alcohol (PVA).

The above prior art reference discloses tablet compositions comprising Ribociclib coated with PVA based coating composition. Still, there exists a need for the development of alternate formulations comprising Ribociclib coated with different coating compositions. The inventors of the present invention have surprisingly found that an immediate release tablet composition comprising Ribociclib coated with a coating composition comprising one or more cellulose derivatives showed comparable/better dissolution with respect to the marketed tablet dosage forms of Ribociclib coated with PVA based coating composition.

### Objective of the Invention

The main objective of the present invention relates to an immediate release pharmaceutical composition comprising Ribociclib or a pharmaceutically acceptable salt thereof.

The present invention also relates to an immediate release tablet composition comprising Ribociclib and one or more pharmaceutically acceptable excipients.

The present invention also relates to a process for the preparation of an immediate release tablet composition comprising Ribociclib and one or more pharmaceutically acceptable excipients having comparable dissolution properties, content uniformity, stability and equivalent bioavailability w.r.t commercialized Ribociclib tablet dosage form.

### SUMMARY OF THE INVENTION

The present invention relates to an immediate release tablet composition comprising Ribociclib or a pharmaceutically acceptable salt thereof and one or more pharmaceutically acceptable excipients.

The present invention also relates to an immediate release tablet composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients, wherein the coating is free of polyvinyl alcohol.

The present invention also relates to a process for the preparation of immediate release composition, comprising the steps of:
(i) blending Ribociclib or a pharmaceutically acceptable salt thereof with one or more pharmaceutically acceptable excipients,
(ii) compressing the blend of step (i) into tablets, and
(iii) coating the tablets obtained in step (ii) with a coating solution comprising one or more cellulose derivatives and one or more pharmaceutically acceptable excipients, wherein the coating is free of polyvinyl alcohol.

### DETAILED DESCRIPTION OF THE INVENTION

The present invention relates to pharmaceutical composition comprising a kinase inhibitor and one or more pharmaceutically acceptable excipients.

The present invention further relates to a pharmaceutical composition comprising Ribociclib or a pharmaceutically acceptable salt thereof and one or more pharmaceutically acceptable excipients.

The present invention further relates to an immediate release pharmaceutical composition comprising Ribociclib or a pharmaceutically acceptable salt thereof and one or more pharmaceutically acceptable excipients.

The present invention further relates to an immediate release tablet composition comprising Ribociclib or a pharmaceutically acceptable salt thereof and one or more pharmaceutically acceptable excipients.

The present invention further relates to an immediate release coated tablet composition comprising Ribociclib or a pharmaceutically acceptable salt thereof and one or more pharmaceutically acceptable excipients.

The present invention further relates to an immediate release coated tablet composition comprising Ribociclib or a pharmaceutically acceptable salt thereof and one or more pharmaceutically acceptable excipients, wherein the coating is free of polyvinyl alcohol.

The present invention also relates to a pharmaceutical composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients.

The present invention also relates to a tablet composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients.

The present invention also relates to an immediate release tablet composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients.

The present invention also relates to a pharmaceutical composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients, wherein the coating is free of polyvinyl alcohol.

The present invention also relates to a tablet composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients, wherein the coating is free of polyvinyl alcohol.

The present invention also relates to an immediate release tablet composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients, wherein the coating is free of polyvinyl alcohol.

In an embodiment, “Ribociclib” according to the present invention includes but not limited to Ribociclib free base and its pharmaceutically acceptable salts, ethers, esters, prodrugs, polymorphs and derivatives thereof.

In another embodiment, the Ribociclib used is in the form of a succinate salt. As used herein, the term “% w/w” refers to the weight of the component based on the total weight of a composition comprising the component.

“Pharmaceutically acceptable excipient/s” are the components added to pharmaceutical formulation to facilitate manufacture, enhance stability, control release, enhance product characteristics, enhance bioavailability, enhance patient acceptability, etc.

In another embodiment, the composition according to the present invention comprises Ribociclib or a pharmaceutically acceptable salt thereof in an amount of 10-90% w/w, preferably 20-80% w/w and more preferably 20-70% w/w of the composition.

In another embodiment, the composition according to the present invention further comprises one or more pharmaceutically acceptable excipients which include but not limited to diluents, disintegrants, binders, surfactants, glidants and lubricants. These excipients may be present intragranularly or extragranularly.

Diluents according to the present invention include but not limited to lactose monohydrate, lactose anhydrous, fructose, maltose, trehalose, dextrose, polydextrose, dextrates, dextrins, isomalt, mannitol, maltitol, xylitol, maltodextrin, lactitol, sorbitol, erythritol, inulin, starch, sucrose, calcium carbonate, dibasic calcium phosphate, tribasic calcium phosphate, calcium sulfate, magnesium carbonate, magnesium oxide, sodium bicarbonate, sodium carbonate, sodium chloride, microcrystalline cellulose, silicified microcrystalline cellulose, cellulose powdered, cellulose acetate, ethyl cellulose, cellulose powdered, kaolin and the like or combinations thereof. The diluent can be used in the range of about 5-90% w/w of the composition.

Binders according to the present invention include but not limited to hydroxypropyl methylcellulose (Hypromellose), hydroxypropyl cellulose, polyvinylpyrrolidone, gelatin, ethyl cellulose, polyvinyl alcohol, pregelatinized starch, carboxymethyl cellulose, sodium alginate, povidone (polyvinyl pyrrolidone), copovidone, microcrystalline cellulose, gelatin, polymethacrylates and the like or combinations thereof. The binder can be used in the range of about 0-40% w/w of the composition.

Disintegrants according to the present invention include but not limited to starches or modified starches such as pregelatinized starch, croscarmellose sodium, carmellose sodium, carmellose calcium, povidone (polyvinyl pyrrolidone), copovidone, crospovidone (Crosslinked polyvinyl pyrrolidone), sodium starch glycolate, low substituted hydroxypropyl cellulose, hydroxypropyl cellulose, alginic acid, and the like or combinations thereof. The disintegrant can be used in the range of about 0-25% w/w of the composition.

Surfactants according to the present invention may be selected from anionic, cationic or non-ionic surface-active agents or surfactants. Suitable anionic surfactants include but not limited to carboxylate, sulfonate, and sulfate ions such as sodium lauryl sulfate (SLS), sodium laurate, dialkyl sodium sulfosuccinates particularly bis-(2-ethylhexyl) sodium sulfosuccinate, sodium stearate, potassium stearate, sodium oleate and the like. Suitable cationic surfactants include but not limited to those containing long chain cations, such as benzalkonium chloride, bis-2-hydroxyethyl oleyl amine or the like. Suitable non-ionic surfactants include but not limited to polyoxyethylene sorbitan fatty acid esters (polysorbates), fatty alcohols such as lauryl, cetyl and stearyl alcohols; glyceryl esters such as the naturally occurring mono-, di-, and tri-glycerides; fatty acid esters of fatty alcohols; polyglycolized glycerides such as gelucire; polyoxyethylene-polyoxypropylene block co-polymer such as Poloxamer and other alcohols such as propylene glycol, polyethylene glycol. The surfactant can be used in the range of about 0-20% w/w of the composition.

Glidants according to the present invention include but not limited to silica, colloidal silicon dioxide, talc and magnesium silicate and mixtures thereof. The glidants can be used in the range of 0-10% w/w of the composition.

Lubricants according to the present invention include but not limited to stearic acid, magnesium stearate, calcium stearate, sodium stearyl fumarate, zinc stearate, glyceryl mono fatty acid, glyceryl monostearate, glyceryl dibehenate, glyceyryl palmito stearic ester, hydrogenated castor oil and mixtures thereof. The Lubricants and/or glidants can be used in the range of 0-10% w/w of the composition.

In another embodiment of the present invention, Ribociclib may be present in crystalline form or amorphous form.

In another embodiment, the present invention provides a pharmaceutical composition comprising Ribociclib or a pharmaceutically acceptable salt thereof in crystalline form and one or more pharmaceutically acceptable excipients.

In another embodiment, the present invention relates to a process for the preparation of immediate release composition, comprising the steps of:
(i) blending Ribociclib or a pharmaceutically acceptable salt thereof with one or more pharmaceutically acceptable excipients,
(ii) compressing the blend of step (i) into tablets, and
(iii) coating the tablets obtained in step (ii) with a coating solution comprising one or more cellulose derivatives and one or more pharmaceutically acceptable excipients.

In another embodiment, the present invention relates to a process for the preparation of immediate release composition, comprising the steps of:
(i) blending Ribociclib or a pharmaceutically acceptable salt thereof with one or more pharmaceutically acceptable excipients,
(ii) compressing the blend of step (i) into tablets, and
(iii) coating the tablets obtained in step (ii) with a coating solution comprising one or more cellulose derivatives and one or more pharmaceutically acceptable excipients, wherein the coating is free of polyvinyl alcohol.

In another embodiment, the present invention relates to a process for the preparation of immediate release composition, comprising the steps of:
(i) blending Ribociclib or a pharmaceutically acceptable salt thereof with one or more pharmaceutically acceptable excipients,
(ii) granulating the blend of step (i),
(iii) blending the granules of step (ii) with one or more pharmaceutically acceptable excipients, and
(iv) lubricating the blend of step (iii) with a lubricant, and
(v) compressing the lubricated material of step (iv) into tablets, and
(vi) coating the tablets obtained in step (v) with a coating solution comprising one or more cellulose derivatives and one or more pharmaceutically acceptable excipients.

In another embodiment, the present invention relates to a process for the preparation of immediate release composition, comprising the steps of:
(i) blending Ribociclib or a pharmaceutically acceptable salt thereof with one or more pharmaceutically acceptable excipients,
(ii) granulating the blend of step (i),
(iii) blending the granules of step (ii) with one or more pharmaceutically acceptable excipients, and
(iv) lubricating the blend of step (iii) with a lubricant, and
(v) compressing the lubricated material of step (iv) into tablets, and
(vi) coating the tablets obtained in step (v) with a coating solution comprising one or more cellulose derivatives and one or more pharmaceutically acceptable excipients, wherein the coating is free of polyvinyl alcohol.

In another embodiment, the present invention relates to a process for the preparation of pharmaceutical composition, comprising the steps of:
(i) dissolving/dispersing Ribociclib or a pharmaceutically acceptable salt thereof with one or more pharmaceutically acceptable excipients in suitable granulating solvents,
(ii) blending one or more pharmaceutically acceptable excipients
(iii) granulating the blend of step (ii) with the solution/dispersion formed in step (i),
(iv) blending the granules of step (iii) with one or more pharmaceutically acceptable excipients, and
(v) lubricating the blend of step (iv) with a lubricant,
(vi) compressing the lubricated material of step (v) into a tablet dosage form, and
(vii) coating the tablets obtained in step (vi) with a coating solution comprising one or more cellulose derivatives and one or more pharmaceutically acceptable excipients.

In another embodiment, the present invention relates to a process for the preparation of pharmaceutical composition, comprising the steps of:
(i) dissolving/dispersing Ribociclib or a pharmaceutically acceptable salt thereof with one or more pharmaceutically acceptable excipients in suitable granulating solvents,
(ii) blending one or more pharmaceutically acceptable excipients
(iii) granulating the blend of step (ii) with the solution/dispersion formed in step (i),
(iv) blending the granules of step (iii) with one or more pharmaceutically acceptable excipients, and
(v) lubricating the blend of step (iv) with a lubricant,
(vi) compressing the lubricated material of step (v) into a tablet dosage form, and
(vii) coating the tablets obtained in step (vi) with a coating solution comprising one or more cellulose derivatives and one or more pharmaceutically acceptable excipients, wherein the coating is free of polyvinyl alcohol.

In another embodiment, the pharmaceutical composition according to the present invention is in the form of tablets, capsules, granules, powder, pellets and sachets.

In another preferred embodiment, the pharmaceutical composition according to the present invention is in the form of tablets.

In another preferred embodiment, the present invention relates to an immediate release tablet composition comprising:
(i) a core composition comprising:
(a) about 10-50% w/w of Ribociclib or a pharmaceutically acceptable salt thereof;
(b) about 40-80% w/w of one or more of diluents;
(c) about 1-20% w/w of one or more disintegrants;
(d) about 0-10% w/w of a one or more glidants,
(e) about 0.1-10% w/w of one or more lubricants,
(ii) a coating composition comprising one or more cellulose derivatives and one or more pharmaceutically acceptable excipients,
wherein the coating is free of polyvinyl alcohol.

In another preferred embodiment, the present invention relates to an immediate release tablet composition comprising:
(i) a core composition comprising:
(a) about 10-50% w/w of Ribociclib or a pharmaceutically acceptable salt thereof;
(b) about 40-80% w/w of one or more of diluents selected from mannitol, microcrystalline cellulose, lactose monohydrate and combination thereof;
(c) about 1-20% w/w of one or more disintegrants selected from sodium starch glycolate, croscarmellose sodium, crospovidone, low substituted hydroxypropyl cellulose and combinations thereof;
(d) about 0-10% w/w of one or more glidant selected from colloidal silicon dioxide, Talc and combinations thereof,
(e) about 0.1-10% w/w of one or more lubricant selected from sodium stearyl fumarate, magnesium stearate and combinations thereof,
(ii) a coating composition comprising one or more cellulose derivatives selected from hydroxypropyl methylcellulose, ethyl cellulose and combination thereof and one or more pharmaceutically acceptable excipients,
wherein the coating is free of polyvinyl alcohol.

In another preferred embodiment, the present invention relates to an immediate release tablet composition comprising:
(i) a core composition comprising:
(a) about 10-50% w/w of Ribociclib or a pharmaceutically acceptable salt thereof;
(b) about 40-80% w/w of one or more of diluents selected from mannitol, microcrystalline cellulose, lactose monohydrate and combination thereof;
(c) about 1-20% w/w of one or more disintegrants selected from sodium starch glycolate, croscarmellose sodium, crospovidone, low substituted hydroxypropyl cellulose and combinations thereof;
(d) about 0-10% w/w of one or more glidant selected from colloidal silicon dioxide, Talc and combinations thereof,
(e) about 0.1-10% w/w of one or more lubricant selected from sodium stearyl fumarate, magnesium stearate and combinations thereof,
(ii) a coating composition comprising one or more cellulose derivatives selected from hydroxypropyl methylcellulose, ethyl cellulose and combination thereof and one or more pharmaceutically acceptable excipients,
wherein the coating is free of polyvinyl alcohol.
wherein the composition is prepared by roller compaction.

In another embodiment, the blend is formulated into a suitable dosage form like tablets or capsules using different techniques which are well known in the prior art.

In another embodiment, the compositions of the present invention may be prepared using any method known in the art, but are not limited to wet granulation, dry granulation, roller compaction, fluidized bed granulation, high-shear granulation, low shear granulation, spray granulation, melt granulation, solid dispersion, encapsulation and direct compression.

In another embodiment, the granulation can be done using one pharmaceutically acceptable excipient, a binder, which can be added to the drug substance in a dissolved state (e.g. in an aqueous/non-aqueous solution) or in a powder form and then granulated by adding a granulation liquid. A combination of more than one binder can be used.

In another embodiment, the solvents used for granulation process may be selected from water, isopropyl alcohol, methanol, ethanol, methylene chloride, dichloromethane or combination thereof.

In another embodiment, the granulation can be done using any method known in the art, but are not limited to fluidized bed granulation, high-shear granulation, low shear granulation, spray granulation and melt granulation.

The pharmaceutical composition may be film coated with functional or non functional layer.

The coating may be selected from amongst one or more of those suitable coating materials known in the art. For example, the coating material can be selected from OPADRY®, INSTAMOIST SHIELD®.

Coating may be performed by applying one or more film forming polymers, with or without other pharmaceutically inert excipients, as a solution/suspension using any conventional coating technique known in the art, such as spray coating in a conventional coating pan or fluidized bed processor; or dip coating.

Coloring agent may be selected from FDA approved colorants such as Iron Oxide, Lake of Tartrazine, Allura Red, Lake of Quinoline Yellow, Lake of Erythrosine, Titanium Dioxide and the like.

In another embodiment, the coating composition comprises one or more cellulose derivatives.

In another embodiment, the cellulose derivatives according to the present invention include but not limited to hydroxypropyl cellulose, hydroxypropyl methylcellulose, ethyl cellulose, methyl cellulose, hydroxy ethyl cellulose, carboxymethyl cellulose, cellulose acetate and combinations thereof.

In one preferred embodiment, the coating composition comprises combination of hydroxypropyl cellulose and ethyl cellulose.

In another preferred embodiment, the coating composition comprises combination of hydroxypropyl methylcellulose and ethyl cellulose.

In yet another embodiment, the present invention provides a tablet composition comprising Ribociclib or a pharmaceutically acceptable salt thereof in the range of about 1 mg to about 500 mg, preferably about 100 mg to about 300 mg and more preferably about 200 mg.

In another embodiment, the present invention provides a tablet composition comprising Ribociclib or a pharmaceutically acceptable salt thereof for the treatment of patients with advanced or metastatic breast cancer.

The invention is further illustrated by the following examples which are provided to be exemplary of the invention and do not limit the scope of the invention.

While the present invention has been described in terms of its specific embodiments, certain modifications and equivalents will be apparent to those skilled in the art and are intended to be included within the scope of the present invention.

### Example 1: Tablet Compositions Comprising Ribociclib

| | | |
|---|---|---|
| | | Quantity |
| S. No | Ingredients | (mg/tab) |

Intra granular

| | | |
|---|---|---|
| 1 | Ribociclib succinate | 254.40* |
| 2 | Microcrystalline cellulose | 377.20 |
| 3 | L-Hydroxypropyl cellulose | 50.00 |
| 4 | Croscarmellose sodium | 25.00 |
| 5 | Colloidal silicon dioxide | 4.00 |
| 6 | Magnesium stearate | 4.20 |

Extra granular

| | | |
|---|---|---|
| 7 | Microcrystalline cellulose | 100.00 |
| 8 | Croscarmellose sodium | 25.00 |
| 9 | Colloidal silicon dioxide | 2.00 |
| 10 | Magnesium stearate | 4.20 |

| | |
|---|---|
| Total weight (Core) | 846.00 |

Coating

| | | |
|---|---|---|
| 11 | Insta Moistshield white | 34.0 |
| | coating composition | |
| 12 | Purified water^{#} | q.s |

| | |
|---|---|
| Total weight (Coated) | 880.00 |

*254.4 mg of Ribociclib succinate is equivalent to 200 mg of Ribociclib
^{#}Does not contribute to the final mass, however present in traces;
q.s—Quantity sufficient

The processing steps involved in manufacturing the tablets were given below:
(i) Ribociclib succinate, microcrystalline cellulose (intra granular), L-Hydroxypropyl cellulose, croscarmellose sodium (intra granular) and colloidal silicon dioxide (intra granular) were sifted and blended,
(ii) the blend obtained in step (i) was lubricated with intra granular magnesium stearate,
(iii) The lubricated blend of step (ii) was roll compacted and milled to obtain granules,
(iv) the granules of step (iii) were mixed with extra granular microcrystalline cellulose, croscarmellose sodium, colloidal silicon dioxide and blended,
(v) the above blend of step (iv) was lubricated with magnesium stearate,
(vi) the lubricated blend of step (v) was compressed into tablets,
(vii) The compressed tablets of step (vi) were coated with a coating suspension which consists of hydroxypropyl cellulose, triacetin, ethyl cellulose, Talc, and titanium dioxide.

### Example 2: Tablet Compositions Comprising Ribociclib

| | | |
|---|---|---|
| | | Quantity |
| S. No | Ingredients | (mg/tab) |

Intra granular

| | | |
|---|---|---|
| 1 | Ribociclib succinate | 254.40* |
| 2 | Microcrystalline cellulose | 67.40 |
| 3 | L-Hydroxypropyl cellulose | 40.00 |
| 4 | Croscarmellose sodium | 15.00 |
| 5 | Colloidal silicon dioxide | 2.00 |
| 6 | Magnesium stearate | 2.10 |

Extra granular

| | | |
|---|---|---|
| 7 | Microcrystalline cellulose | 25.00 |
| 8 | Croscarmellose sodium | 15.00 |
| 9 | Colloidal silicon dioxide | 1.00 |
| 10 | Magnesium stearate | 2.10 |

| | |
|---|---|
| Total weight (Core) | 424.00 |

Coating

| | | |
|---|---|---|
| 11 | Insta Moistshield white | 16.0 |
| | coating composition | |
| 12 | Purified water^{#} | q.s |

| | |
|---|---|
| Total weight (Coated) | 440.00 |

*254.4 mg of Ribociclib succinate is equivalent to 200 mg of Ribociclib
^{#}Does not contribute to the final mass, however present in traces;
q.s—Quantity sufficient

The processing steps involved in manufacturing the Ribociclib tablets is similar to the process provided in Example 1.

### Example 3: Tablet Compositions Comprising Ribociclib

| | | |
|---|---|---|
| | | Quantity |
| S. No | Ingredients | (mg/tab) |

Intra granular

| | | |
|---|---|---|
| 1 | Ribociclib succinate | 254.40* |
| 2 | Microcrystalline cellulose | 377.20 |
| 3 | L-Hydroxypropyl cellulose | 50.00 |
| 4 | Croscarmellose sodium | 25.00 |
| 5 | Colloidal silicon dioxide | 4.00 |
| 6 | Purified water^{#} | q.s |

Extra granular

| | | |
|---|---|---|
| 7 | Microcrystalline cellulose | 100.00 |
| 8 | Croscarmellose sodium | 25.00 |
| 9 | Colloidal silicon dioxide | 2.00 |
| 10 | Magnesium stearate | 8.40 |
| | Total weight (Core) | 846.00 |

Coating

| | | |
|---|---|---|
| 11 | Insta Moistshield white | 34.0 |
| | coating composition | |
| 12 | Purified water^{#} | q.s |

| | |
|---|---|
| Total weight (Coated) | 880.00 |

*254.4 mg of Ribociclib succinate is equivalent to 200 mg of Ribociclib
^{#}Does not contribute to the final mass, however present in traces;
q.s—Quantity sufficient

The processing steps involved in manufacturing the tablets were given below:
(i) Ribociclib succinate, microcrystalline cellulose (intra granular), L-Hydroxypropyl cellulose, croscarmellose sodium (intra granular) and colloidal silicon dioxide (intra granular) were sifted and blended,
(ii) the blend obtained in step (i) was granulated using purified water as granulating solvent and the granules were dried and sifted,
(iii) the granules obtained in step (ii) were mixed with extra granular microcrystalline cellulose, croscarmellose sodium, colloidal silicon dioxide and blended,
(iv) the above blend of step (iii) was lubricated with magnesium stearate,
(v) the lubricated blend of step (iv) was compressed into tablets,
(vi) The compressed tablets of step (v) were coated with a coating suspension which consists of hydroxypropyl cellulose, triacetin, ethyl cellulose, Talc, and titanium dioxide.

### Example 4: Tablet Compositions Comprising Ribociclib

| | | |
|---|---|---|
| | | Quantity |
| S. No | Ingredients | (mg/tab) |

Intra granular

| | | |
|---|---|---|
| 1 | Ribociclib succinate | 254.40* |
| 2 | Microcrystalline cellulose | 67.40 |
| 3 | L-Hydroxypropyl cellulose | 40.00 |
| 4 | Croscarmellose sodium | 15.00 |
| 5 | Colloidal silicon dioxide | 2.00 |
| 6 | Purified water^{#} | q.s |

Extra granular

| | | |
|---|---|---|
| 7 | Microcrystalline cellulose | 25.00 |
| 8 | Croscarmellose sodium | 15.00 |
| 9 | Colloidal silicon dioxide | 1.00 |
| 10 | Magnesium stearate | 4.20 |

| | |
|---|---|
| Total weight (Core) | 424.00 |

Coating

| | | |
|---|---|---|
| 11 | Insta Moistshield white | 16.00 |
| | coating composition | |
| 12 | Purified water^{#} | q.s |

| | |
|---|---|
| Total weight (Coated) | 440.00 |

*254.4 mg of Ribociclib succinate is equivalent to 200 mg of Ribociclib
^{#}Does not contribute to the final mass, however present in traces;
q.s—Quantity sufficient

The processing steps involved in manufacturing the Ribociclib tablets is similar to the process provided in Example 3.

### Example 5: Tablet Compositions Comprising Ribociclib

| | | |
|---|---|---|
| | | Quantity |
| S. No | Ingredients | (mg/tab) |

Intra granular

| | | |
|---|---|---|
| 1 | Ribociclib succinate | 254.40* |
| 2 | Mannitol | 220.35 |
| 3 | Microcrystalline cellulose | 73.45 |
| 4 | L-Hydroxypropyl Cellulose | 20.0 |
| 5 | Croscarmellose sodium | 10.0 |
| 6 | Sodium Stearyl Fumarate | 3.65 |

Extra granular

| | | |
|---|---|---|
| 7 | Microcrystalline cellulose | 120.85 |
| 8 | Croscarmellose sodium | 20.0 |
| 9 | Sodium Stearyl Fumarate | 7.3 |

| | |
|---|---|
| Total weight (Core) | 730.00 |

Coating

| | | |
|---|---|---|
| 11 | Insta Moistshield ® Purple | 25.50 |
| | A21G01605^{@} | |
| 12 | Purified water^{#} | q.s |

| | |
|---|---|
| Total weight (Coated) | 755.50 |

*254.4 mg of Ribociclib succinate is equivalent to 200 mg of Ribociclib
^{#}Does not contribute to the final mass, however present in traces;
@Insta Moistshield purple A21G01605 contains Hypromellose, Triacetin, Ethyl cellulose, Talc, Titanium dioxide, Black iron oxide and Red iron oxide
q.s—Quantity sufficient

The processing steps involved in manufacturing the tablets were given below
i. Ribociclib succinate, Microcrystalline cellulose (intra granular), Mannitol, L-Hydroxypropyl Cellulose and Crocarmellose sodium (intra granular) were sifted and blended,
ii. The blend obtained in step (i) was lubricated with intra granular sodium Stearyl fumarate,
iii. The lubricated blend of step (ii) was roll compacted and milled to obtain granules,
iv. The granules of step (iii) were mixed with extra granular microcrystalline cellulose and croscarmellose sodium and blended,
v. The above blend of step (iv) was lubricated with sodium Stearyl fumarate,
vi. The lubricated blend of step (v) was compressed into tablets, and
vii. The compressed tablets of step (vi) were coated with Insta Moistshield purple coating suspension which consists of Hypromellose, Triacetin, Ethyl cellulose, Talc, Titanium dioxide, Black iron oxide and Red iron oxide.

We claim:

1. An immediate release tablet composition comprising:
(i) a core comprising Ribociclib or a pharmaceutically acceptable salt thereof, and one or more pharmaceutically acceptable excipients; and
(ii) a coating composition comprising one or more cellulose derivatives and one or more pharmaceutically acceptable excipients,
wherein the coating is free of polyvinyl alcohol.

2. The tablet composition as claimed in claim 1, wherein one or more pharmaceutically acceptable excipients are selected from diluents, disintegrants, binders, surfactants, glidants and lubricants.

3. The tablet composition as claimed in claim 2, wherein the diluent is selected from lactose monohydrate, lactose anhydrous, fructose, maltose, trehalose, dextrose, polydextrose, dextrates, dextrins, isomalt, mannitol, maltitol, xylitol, maltodextrin, lactitol, sorbitol, erythritol, inulin, starch, sucrose, calcium carbonate, dibasic calcium phosphate, tribasic calcium phosphate, calcium sulfate, magnesium carbonate, magnesium oxide, sodium bicarbonate, sodium carbonate, sodium chloride, microcrystalline cellulose, silicified microcrystalline cellulose, cellulose powdered, cellulose acetate, ethyl cellulose, cellulose powdered, kaolin and combination thereof.

4. The tablet composition as claimed in claim 2, wherein the disintegrant is selected from starch, modified starches, pregelatinized starch, croscarmellose sodium, carmellose sodium, carmellose calcium, povidone, copovidone, crospovidone, sodium starch glycolate, low substituted hydroxypropyl cellulose, hydroxypropyl cellulose, alginic acid and combination thereof.

5. The tablet composition as claimed in claim 1, wherein one or more cellulose derivatives are selected from hydroxypropyl cellulose, hydroxypropyl methylcellulose, ethyl cellulose, methyl cellulose, hydroxyethyl cellulose, carboxymethyl cellulose, cellulose acetate and combination thereof.

6. A process for the preparation of composition as claimed in claim 1, comprising the steps of:
(i) blending Ribociclib or a pharmaceutically acceptable salt thereof with one or more pharmaceutically acceptable excipients,
(ii) compressing the blend of step (i) into tablets, and
(iii) coating the tablets obtained in step (ii) with a coating solution comprising one or more cellulose derivatives and one or more pharmaceutically acceptable excipients,
wherein the coating is free of polyvinyl alcohol.

7. The process for the preparation of tablet composition as claimed in claim 6, wherein the process is selected from wet granulation, dry granulation, roller compaction, fluidized bed granulation, high-shear granulation, low shear granulation, spray granulation, melt granulation and direct compression.

8. A process for the preparation of tablet composition as claimed in claim 1, comprising the steps of:
(i) blending Ribociclib or a pharmaceutically acceptable salt thereof with one or more pharmaceutically acceptable excipients,
(ii) granulating the blend of step (i),
(iii) blending the granules of step (ii) with one or more pharmaceutically acceptable excipients, and
(iv) lubricating the blend of step (iii) with a lubricant, and
(v) compressing the lubricated material of step (iv) into tablets, and
(vi) coating the tablets obtained in step (v) with a coating solution comprising one or more cellulose derivatives and one or more pharmaceutically acceptable excipients,
wherein the coating is free of polyvinyl alcohol.

9. The tablet composition as claimed in claim 1 comprising:
(i) a core composition comprising:
(a) about 10-50% w/w of Ribociclib or a pharmaceutically acceptable salt thereof;
(b) about 40-80% w/w of one or more of diluents selected from mannitol, microcrystalline cellulose, lactose monohydrate and combination thereof;
(c) about 1-20% w/w of one or more disintegrants selected from sodium starch glycolate, croscarmellose sodium, crospovidone, low substituted hydroxypropyl cellulose and combinations thereof;
(d) about 0-10% w/w of one or more glidant selected from colloidal silicon dioxide, Talc and combinations thereof,
(e) about 0.1-10% w/w of one or more lubricant selected from sodium stearyl fumarate, magnesium stearate and combinations thereof,
(ii) a coating composition comprising one or more cellulose derivatives selected from hydroxypropyl methylcellulose, ethyl cellulose and combination thereof and one or more pharmaceutically acceptable excipients,
wherein the coating is free of polyvinyl alcohol,
wherein the composition is prepared by roller compaction.
